(19) United States
(12) Patent Application Publication
(10) Pub. No.: US 2004/0053938 A1
(43) Pub. Date: Mar. 18, 2004

(54) Novel method and device for treatment of exercise induced pulmonary hemorrhage in horses

(76) Inventors: Calvert R. Bratton, Frankfort, KY (US); Thomas Tobin, Lexington, KY (US)

Correspondence Address: STOCKWELL & ASSOCIATES, PSC, 861 CORPORATE DRIVE, SUITE 201, LEXINGTON, KY, 40503, US

(21) Appl. No.: 10/466,803

(22) PCT Filed: Aug. 1, 2002
(86) PCT No.: PCT/US02/24588

(51) Int. Cl. A61K 31/519

(52) U.S. Cl. 514/252.16

(57) ABSTRACT

A device and method are provided for convenient, patient compliant inhalation therapy of equine species. The device can be used to deliver any drug or other pharmaceutical agent which can be adapted for inhalation directly into the nasal passages of a horse and thereby provide inhalation therapy with minimal discomfort. The device can be used to treat any of a number of conditions including, EIPH, infections and allergies, e.g., asthma or heaves. In one embodiment, the invention provides a method for the treatment or prevention of EIPH, utilizing a composition adapted for inhalant therapy comprised of sildenafil citrate alone or in combination other agents.

This application claims the benefit of priority in U.S. Provisional Application Serial No. 60/309389 filed on Aug. 1, 2001.

### FIELD OF THE INVENTION

The present invention generally relates to methods and devices for inhalation therapy in horses. In particular, the invention relates to methods and devices for nebulization therapy in horses for the treatment and prevention of respiratory conditions including Exercise-Induced Pulmonary Hemorrhage (EIPH) and related pulmonary sequella in the horse.

### BACKGROUND OF THE INVENTION

Exercise Induced Pulmonary Hemorrhage (EIPH) is an endemic production disease of racing and other high-intensity exercise horses. EIPH or “bleeding” has been a recognized condition in racing horses for at least three hundred years. Virtually all horses that are subjected to intense exercise bleed into the lungs, and these episodes of bleeding often commence as soon as these horses enter training. Healing occurs, but complete restoration of pulmonary function in the affected area often does not occur. Repeated episodes of intense exercise can result in repeated episodes of pulmonary hemorrhage, and cumulative damage to the affected lung tissue can occur such as e.g., fibrosis and/or scaring and consolidation of alveoli. These chronic changes occur, particularly in the dorso-caudal lobes of the lung, and such changes can eventually curtail the performance of the horse.

Preventative/ameliorative/curative/restorative measures for EIPH affected horses have also been sought for several hundred years. For many years, the treatment of choice for prevention of EIPH in the race horse has been pre-race treatment with the diuretic furosamide (Lasix®). The exact mechanism of action of furosamide in prevention of EIPH is unknown, although many theories have been postulated over the years. The treatment of choice for EIPH, after the fact, is usually rest (mandatory in many racing jurisdictions) and often in conjunction with antibiotics to prevent secondary bacterial infection and/or the use of anti-inflammatory medication.

More recently, (following the research of West et al. J. Appl. Physiol. 1993, 75: 1097-1109 related to the relationship of EIPH and increased pulmonary artery pressure) attempts at treating EIPH via nitric oxide administration have been tried, e.g., by Perry (U.S. Pat. No. 5,765,548). Perry describes administration of nitric oxide through continuous insufflation of the nitric oxide to the horse during the exercise period. Alternatively, the horse is treated with insufflation of nitric oxide prior to the exercise event and then is given an intramuscular injection of a phosphodiesterase inhibitor, e.g., ZAPRINAST. The treatment during exercise as described by Perry is both cumbersome and problematic for the racing animal and has never gained widespread acceptance. Likewise, systemic treatment of the racing animal with phosphodiesterase inhibitors opens the door for unwanted side effects and requires regulatory scrutiny.

Thus, prior to the present invention, there has not been a safe and effective method to prevent and/or treat EIPH in the racing animal. Accordingly, there still exists a need in the art for such methods.

### OBJECTS OF THE INVENTION

It is an object of the invention to provide an inhalation therapy regimen and system for the treatment and/or prevention of EIPH comprising the inhalation of a therapeutic amount of sildenafil citrate and/or the metabolites of sildenafil citrate alone or in combination with other compounds.

It is a further object of the invention to provide a device suitable for inhalation delivery of a pulmonary therapeutic agent, e.g., delivery through an equine nasal cannula into the nasal passages of an equine.

Another object of the invention is to provide a device comprised of an equine nasal cannula designed to place the therapeutic inhalant directly into the nasal passages without covering the nasal openings of the subject equine thereby allowing reasonable mobility and reducing patient discomfort while undergoing therapy and thereby improve patient acceptance.

Thus, a primary object of the invention is to provide an improved apparatus or device for delivery of an inhaled therapeutic regimen into the nasal passages of an equine wherein said device is comfortable for the equine and is also easy to visually inspect and maintain when in use.

Still another object of the invention is to provide prophylactic and therapeutic benefits of sildenafil citrate, or the metabolites of sildenafil citrate, as a nebulized inhalant inducing the local release of nitric oxide or precursors or donors. Sildenafil citrate and/or its metabolites, salts, isomers and/or derivatives thereof, used according to the invention may be administered alone or in combination with other agents, in the prophylaxis, treatment, and resolution of the pathological changes associated with Exercise Induced Pulmonary Hemorrhage (EIPH) in horses.

### SUMMARY OF THE INVENTION

In the field of veterinary medicine, the equine athlete and particularly the racing equine, may, and many indeed will, develop Exercise Induced Pulmonary Hemorrhage (EIPH) due to the training and exercise requirements needed to effectively compete in athletic events. The current invention provides a method and device for the administration of single or combined therapeutic elements, e.g., comprised of sildenafil citrate and other components, to provide an effective remedy for EIPH.

The device of the invention allows for the uninterrupted administration of a specified therapeutic regimen in such a manner that the subject equine is able to maintain adequate mobility during therapy, e.g., to move about a stall or to allow access to food and water while continuously receiving therapy. Therapy may generally be administered before and/or after exercise, training or exertion. The concentration of the active comprising the therapeutic mixture and the rate of delivery can, of course, vary according to the apparent physical condition (e.g., size of the animal and severity of EIPH) and specific needs of each individual equine.

Thus, the present invention provides an inhalation therapy regimen administered through, e.g., a non-invasive equine nasal cannula device as also provided by the invention. In one embodiment, the therapy regimen includes the introduction of a composition comprised of sildenafil citrate or congeners of sildenafil citrate with or without potentiating agents delivered to the subject equine via the attendant inhalation therapy system in a non-invasive manner that is conducive to the care and comfort of the equine mammal under treatment. In one embodiment the composition comprised of sildenafil citrate is nebulized for delivery as an inhalant into the pulmonary system of the subject equine.

### BRIEF DESCRIPTION OF THE DRAWINGS

FIG. 1 is a schematic representation of the device of the invention in use on a horse. Bilateral nasal cannulas can be seen in a desired position adjacent the horse's nostrils for delivery of a therapeutic inhalant agent.

FIG. 2 is a top plan view of one embodiment of the device of the invention.

FIG. 3 is a perspective view of one embodiment of the nasal cannula portion of the device of the invention.

FIG. 4 is a perspective view of one embodiment of the inhalator manifold of the device of the invention.

FIG. 5 is a perspective view of one embodiment of the nebulization apparatus and air supply for delivery of a desired therapeutic agent via the device of FIGS. 1-2.

FIG. 6 is a color digital photograph showing a left elevational view of one embodiment of the device of the invention.

FIG. 7 is a color digital photograph showing a left frontal view of one embodiment of the device of the invention.

FIG. 8 is a color digital photograph showing a top plan view of one embodiment of the device of the invention.

FIG. 9 is a perspective view of an alternate embodiment of the inhalator manifold shown in FIG. 4.

### DETAILED DESCRIPTION OF THE INVENTION

The foregoing and other objects, features and advantages of the present invention should become apparent to those of skill in the art in the following description when taken in conjunction with the accompanying drawings and through practice of the invention.

The present invention provides a device and method for convenient, patient compliant inhalation therapy of equine species. The device of the invention can be used to deliver any agent to the nostrils or nasal passages of a horse for inhalation therapy and can be used to treat any of a number of conditions including, but not limited to e.g., pneumonia (bacterial and viral mediated), asthma or heaves and the like. However, a preferred utilization of the device as set forth herein is in the treatment and/or prevention of EIPH and related pulmonary conditions in horses.

The inhalation therapy device and regimen provided herein comprises a non-invasive equine nasal cannula developed exclusively to deliver an inhalant therapy mixture primarily for use in the treatment of equine exercise induced pulmonary hemorrhage (EIPH) and related ailments. As can be appreciated by one of skill in the art, the equine nasal cannula (the device) of the invention can also be used for delivery of any mendicant suitable for or adapted for inhalant delivery including, but not limited to the simple placement of oxygen into the nasal passages of equine mammals or any of a number of inhalant delivery forms known in the art, e.g., via nebulization of the active.

The Equine Nasal Cannula

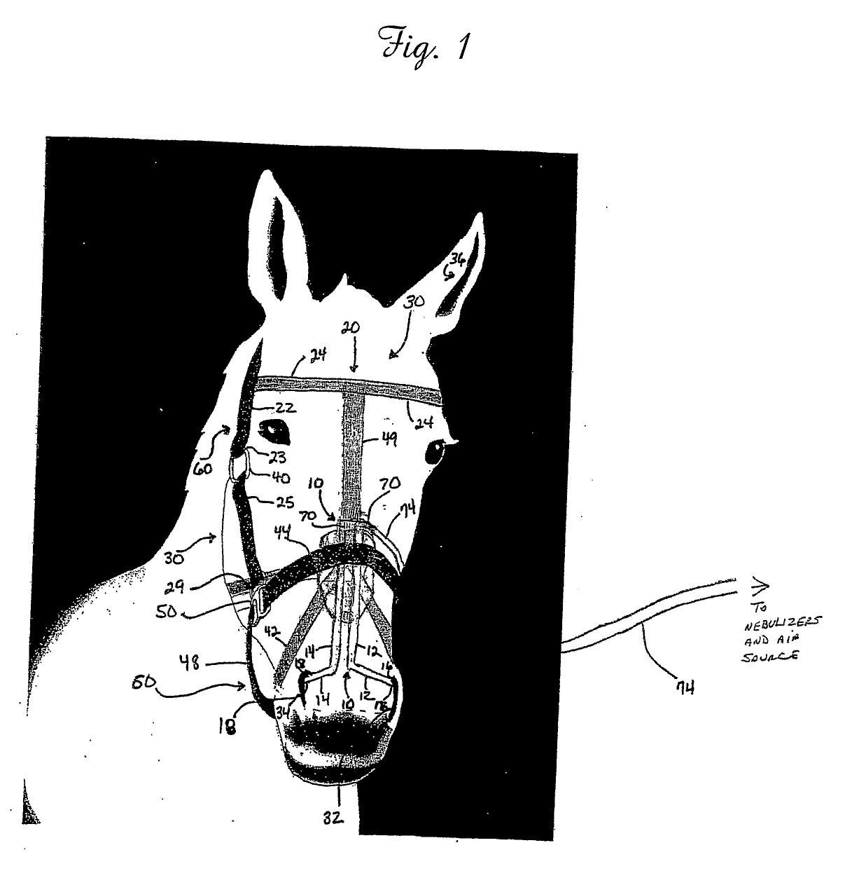

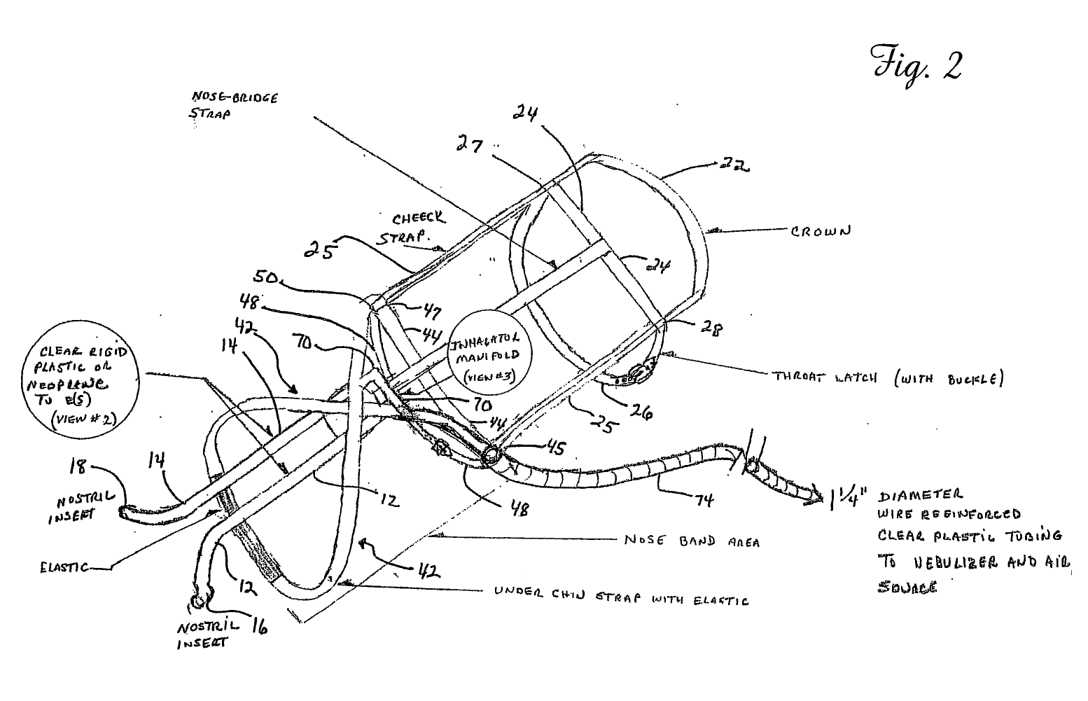

As shown in FIG. 1, one embodiment of the device 10 of the invention is an equine nasal cannula that is designed with means 20 for removable attachment to the head 30 of the subject equine. In the embodiment shown, in FIG. 1, the device 10 and attachment means 20 are shown attached to the head 30 of the subject equine and lying beneath a standard equine halter 60. Thus, one of skill in the art can appreciate that the device 10 and attachment means 20 can be attached and used independent of but in conjunction with halters, bridles or other equine head gear. Alternatively, the attachment means 20 can be connected directly to or integral with (incorporated into, e.g., a halter of the type shown in FIG. 1 (halter 60) or as shown in FIG. 2.

In the embodiment shown in FIG. 1, the attachment means 20 itself is a halter-type or halter-like apparatus constructed of nylon or leather with adjustable straps for securing to the head 30 of the subject equine and is separate from halter 60. As set forth more filly below, the attachment means 20 for the device 10 can be separate from halter 60 or integrated therein. As can be appreciated by one of skill int the art, the attachment means can be constructed from other suitable materials including, but not limited to plastic, rope and the like.

The device 10 is held in position by attachment means 20 such that the distal ends 16, 18 of elongated delivery tubes 12, 14 (cannulas) are placed adjacent the external openings 32, 34 of the subject equine's nasal cavity (adjacent the nares) so as to effectively deliver the composition selected for inhalation at the appropriate site. In a preferred embodiment, the methods of the invention provide for delivery of a preselected active ingredient via device 10 in an inhaled therapeutic regimen comprised of at least one active delivered directly into the apertures of the equine's nasal passages in such a manner as to allow the inhalation of substantially the entire volume of the therapeutic composition delivered to the equine's nasal passages.

In the presently preferred embodiment, the device 10 of the invention (the equine nasal cannula) is comprised of a nylon attachment means 20 (similar to a halter) that is configured to adapt to the contours of the equine skull 30 in such a manner as to effectively place and hold the distal ends 16, 18 of the elongated delivery tubes 12, 14 in a desired position for the administration of a therapeutic inhalant while minimizing the requirements for specialized animal restraint and/or restriction of movement.

This device 10 (equine nasal cannula) is designed in such a way as to place the inhalation therapy stream, e.g., a nebulized composition or simply oxygen directly into the nasal apertures 32, 34 (adjacent the mucocutaneous junction of the nares) without entering or occluding or otherwise covering the nasal passages. This device and protocol also has the advantage of reducing the tendency for the nasal passages to become dry and irritated during therapy.

In one embodiment, padded extrusions (not shown) are provided at the distal ends 16, 18 (nasal end) of the elongated tubes 12, 14 (cannulas) to prevent injury or irritation that may occur from rubbing the nostrils or the inner linings (mucosa) of the nostrils. The padded extrusions can be designed with slots on the sides or holes or other such configurations to enable the flow of the therapy stream to continue in the event a portion of the cannula outlet becomes blocked or clogged with mucus or from exhaled environmental contaminates.

The device 10 or equine nasal cannula can be designed to conform to or become an integral part of harness and/or bridle or other halter-type equipment normally associated with the training and racing of equine athletes. The device 10 or equine nasal cannula is user friendly in that it closely resembles equipment that racing animals are familiar with. Further, the device 10 or equine nasal cannula is easy to position on the equine animal's head 30 and is easily adjustable to fit nearly any equine. When constructed of a suitable material such as nylon, plastic or leather, it is also easy to care for and clean.

FIG. 1 is a perspective view of an equine head 30 fitted with the device 10 (equine nasal cannula) (showing the device in use) e.g., preparatory to receiving a composition comprised of sildenafil citrate and/or its congeners, potentiating agents, complementing agents and carrier gases for the treatment or prevention of EIPH.

While the device 10 (equine nasal cannula) in the illustrations contained herein has specific dimensional and other characteristics (see, FIGS. 2-5.), one of skill in the art will appreciate that other variations are possible and are within the scope of the invention. Thus, the dimensional and other characteristics shown in FIGS. 2-5 are in no way intended as limitations and are only illustrative of one embodiment of the present invention.

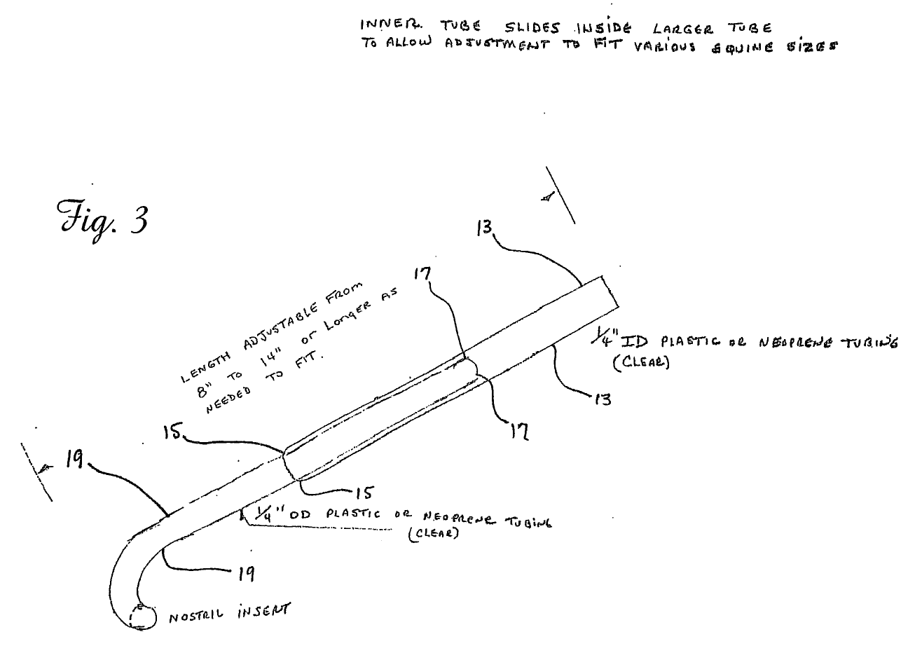

In a preferred embodiment shown in FIG. 3, the elongated tubes (shown as 12, 14 in FIG. 1) are adjustable and may be adapted for use with different size horses i.e., for lengthening or shortening the tube length so as to provide the proper distance or length of tube such that distal ends 16, 18 are properly positioned at the external opening of the equine's nose 32, 34 thereby directing a fluid flowing there through directly into the equine nasal openings. In the embodiment shown in FIG. 3, the adjustment means for elongated tubes 12, 14 comprises a first segment or tube 13 having a distal end 15 with an inside diameter that is sized complimentary to the outside diameter of the proximal end 17 of a second segment or tube 19 such that the proximal end 17 of the second segment 19 is slidably and sealingly engaged with the distal end 15 of the first segment 13.

As illustrated in FIG. 3 and by way of example, one embodiment of the invention comprises two segments of clear plastic or neoprene elongated tubing 13,19 wherein the first segment 13 is comprised of a ¼ inch inside diameter (I.D.) and the second segment 19 comprises a ¼ inch outside diameter (O.D.) whereby proximal end 17 of the second segment 19 is slidably and sealingly engaged within the distal end 15 of first segment 13. A presently preferred embodiment of the invention however, comprises two segments of clear plastic or neoprene elongated tubing 13,19 as shown in FIG. 3 wherein the first segment 13 is comprised of a 5/16th inch inside diameter (I.D.) and the second segment 19 comprises a 5/16th inch outside diameter (O.D.) whereby proximal end 17 of the second segment 19 is slidably and sealingly engaged within the distal end 15 of first segment 13. Of course, one of skill in the art can appreciate that other configurations, sizes of tubing and choice of materials are possible and are within the scope of the invention.

Referring now to the attachment means 20, shown in the embodiment set forth in FIGS. 1-2, another embodiment contemplated by the invention is to have the attachment means 20 incorporated into or integral with halter 60. In this alternate embodiment, the upper portion of the halter-like structure can have an elastic or other adjustable band, the crown strap 22 which is attached to buckles 40 at distal ends 23 and designed so as to fit the subject equine's head 30 behind the ears 36. Crown strap 22 can further comprise adjustable fasteners (not shown) at for attaching to buckles 40 for adjustment of crown strap 22 to fit different sized heads 30.

Forehead strap 24 can slidably attach to crown strap 22 adjacent to or confluent with adjustable throat latch 26 which works in conjunction with the crown strap 22 to secure the upper portion of attachment means 20 to the head 30. Optionally, the forehead strap 24 can attach to buckles 40 depending upon the desired length of crown strap 22. Cheek straps 25 are connected to buckles 40 at the proximal ends 27, 28 thereof and can be connected to the nose band assembly 42 at the distal ends of cheek strap 25.

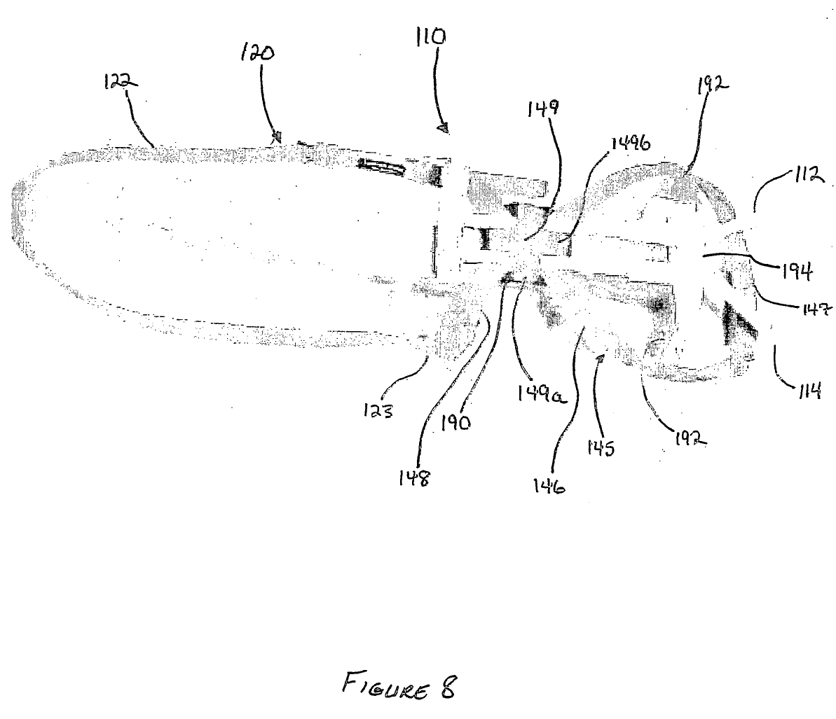

Still referring to FIGS. 1-5, the lower portion of the halter-like configuration of attachment means 20 of device 10 can be configured to surround the mandibles 33 of the head 30 to hold the elongated delivery tubes 12, 14 firmly in place on the bridge of the equine's nose. For example, a portion of the lower section may contain an elastic nose band assembly 42 (with/or without adjustable fasteners) designed in a “figure 8” arrangement so as to fit firmly under the throat latch, over the bridge of the nose and under the chin such that the strap of the figure-8 loop crosses over the bridge of the nose thereby enabling elongated tubes 12, 14 of the cannula device cannula to be held in place throughout the therapy regimen. Nose band assembly 42 which comprises the lower portion of attachment means 20 can be connected to the upper portion of attachment means 20 by nose bridge strap 49. The proximal end of nose bridge strap 49 attaches to forehead strap 24 and attaches at its distal end to nose band 42.

In the embodiment shown in FIGS. 1-2, the nose band assembly 42 can optionally further comprise a bridge band 44 having distal ends 45, 47 designed to fit over the bridge of the equine's nose and attach to a triple loop buckle assembly (such as is shown in FIG. 1 at 50) at proximal and distal ends 45, 47 of bridge band 44. Adjustable chin strap 48 may also be connected to triple loop buckle assembly 50 to give additional support and security to the attachment means 20. It can be appreciated that buckle the triple loop buckle assembly 50 can have a variety of configurations varying from the ring like structures shown in FIG. 1 to other configurations known in the art.

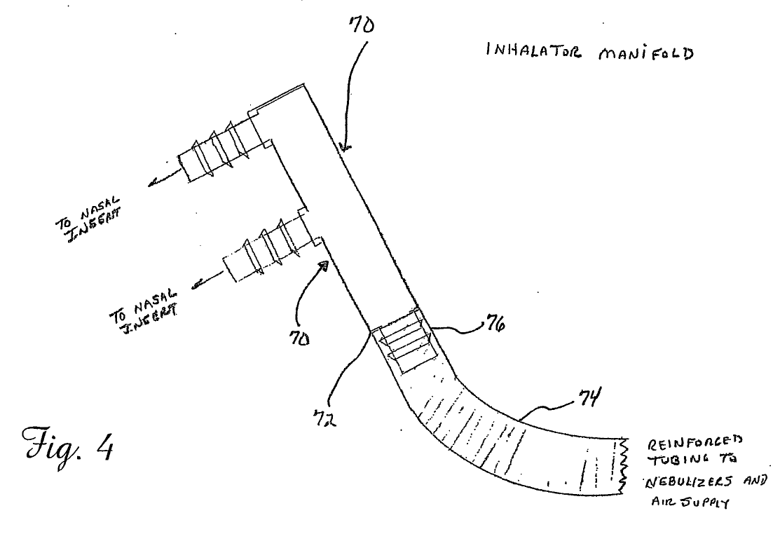

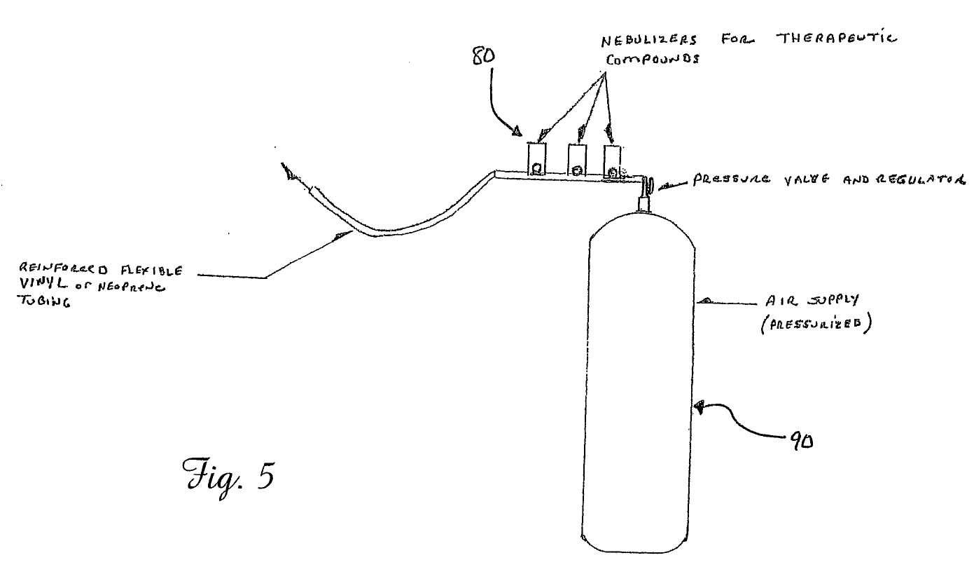

FIG. 4 is an isolated view of an inhalator manifold 70 which can be used to connect the remote drug supply means, e.g., the nebulizers 80 and pressurized air supply 90 shown in FIG. 5 and facilitate delivery of the active agent to the desired site. The inhalator manifold 70 can be constructed of any suitable material including metals such as aluminum, stainless steel, plastics and the like. The proximal end 72 of inhalator manifold 70 is adapted for a removable connection to air supply tube 74 at its distal end 76. Preferably, the air supply tube 74 is flexible, elastic and constructed of a reinforced material such as rubber or plastic. In one embodiment, the air supply tube 74 is a ¼ inch flexible wire reinforced clear plastic tubing. The proximal end (not shown) of flexible tube 74 connects to the air supply and drug source. The distal end 77 of inhalator manifold 70 contains a means for sealing yet removable connection to elongated tubes 12, 14 as shown in FIGS. 1-2 and FIGS. 6-8.

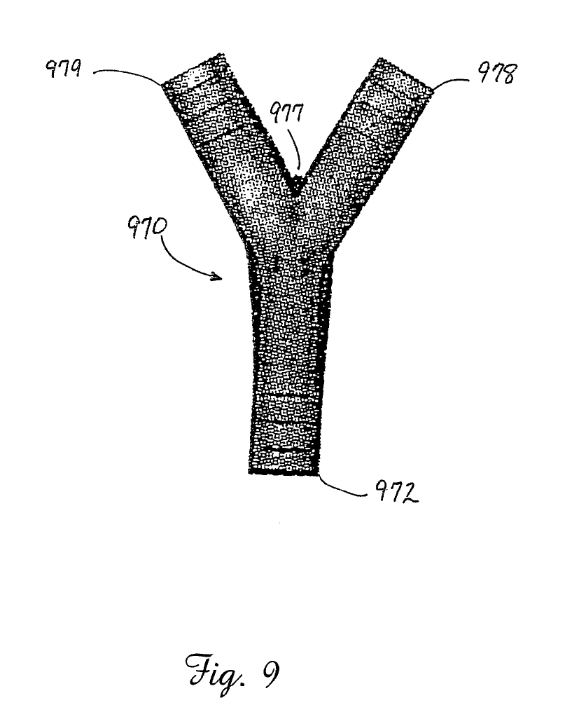

A presently preferred embodiment of the inhalator manifold is shown in FIG. 9. FIG. 9 is an isolated perspective view of inhalator manifold 970 and is an alternate embodiment of the inhalator manifold 70 shown in FIGS. 1& 4. Inhalator manifold 970 can be used to connect the remote drug supply means, e.g., the nebulizers 80 and pressurized air supply 90 shown in FIG. 5 to elongated tubes 12, 14 as shown in FIGS. 1-2 and FIGS. 6-8. The inhalator manifold 970 can be constructed of any suitable material including metals such as aluminum, stainless steel, plastics and the like. The proximal end 972 of inhalator manifold 970 is adapted for a removable connection to the air supply tube (e.g., air supply tube 74 shown in FIG. 4). The proximal end (not shown) of the flexible air supply tube 74 in turn connects to the air supply and drug source (not shown). The distal end 977 of inhalator manifold 970 contains a means for sealing yet removable connection to elongated tubes or nasal cannulas of the type shown as elongated tubes 12, 14 in FIGS. 1-2 and FIGS. 6-8.

In the embodiment shown in FIG. 9, distal end 977 of inhalator manifold 970 forms a “Y” bifurcation terminating in distal connecting ends 978,979 which are adapted for removable connection to the proximal ends of elongated tubes 12,14 shown in FIG. 1. Thus, fluid flow through the device will originate from an outside air supply (e.g., air supply 90 as shown in FIG. 5) and may pass through a nebulizer 80 for addition of an active agent or drug and into air supply tube 74. The fluid composition containing the nebulized active then passes through air supply tube 74 into the proximal end 972 of the inhalator manifold 970, through the “Y” bifurcation and distal connecting ends 978,979 and into elongated tubes (nasal cannulas) 12,14 for delivery into the equine nasal passages. The improved airflow properties of the “Y” bifurcation design of inhalator manifold 970 acts to lessen or reduce any tendency for the nebulized or atomized active agent to accumulate within the manifold. The inhalator manifold 970 also aids in providing the necessary airflow volume for delivery of various active agents, e.g., airflow volumes of between about 2-2.5 liters of air per minute. In one embodiment, the inside diameter of inhalator manifold 970 is about ⅝th of an inch to accommodate the desired airflow volumes.

In general, the dorsal portion of the lower section of the attachment means of the device (the equine nasal cannula) is designed to provide for the placement of the distal ends of two elongated plastic tubes into or adjacent to the nasal apertures (openings) of the equine mammal nasal passages.

The elongated plastic tubes are attached to the dorsal portion of the lower section of the attachment means in a manner allowing for movement of the tubes in two directions to enable the nasal portions of the tubes to be adjusted to fit the majority of equine mammals.

The elongated plastic tubes are connected to flexible plastic tubing via a manifold in such a manner as to provide firm but removable attachment to the thereto. This flexible plastic tubing connects the equine nasal cannula to the therapy supply at the source of the therapeutic inhalant or oxygen outside the confinement area of the equine.

The portion of flexible tubing extending from the cannula connection to the therapy supply can be wire spring reinforced to prevent collapse or the disruption of the therapy supply to the cannula.

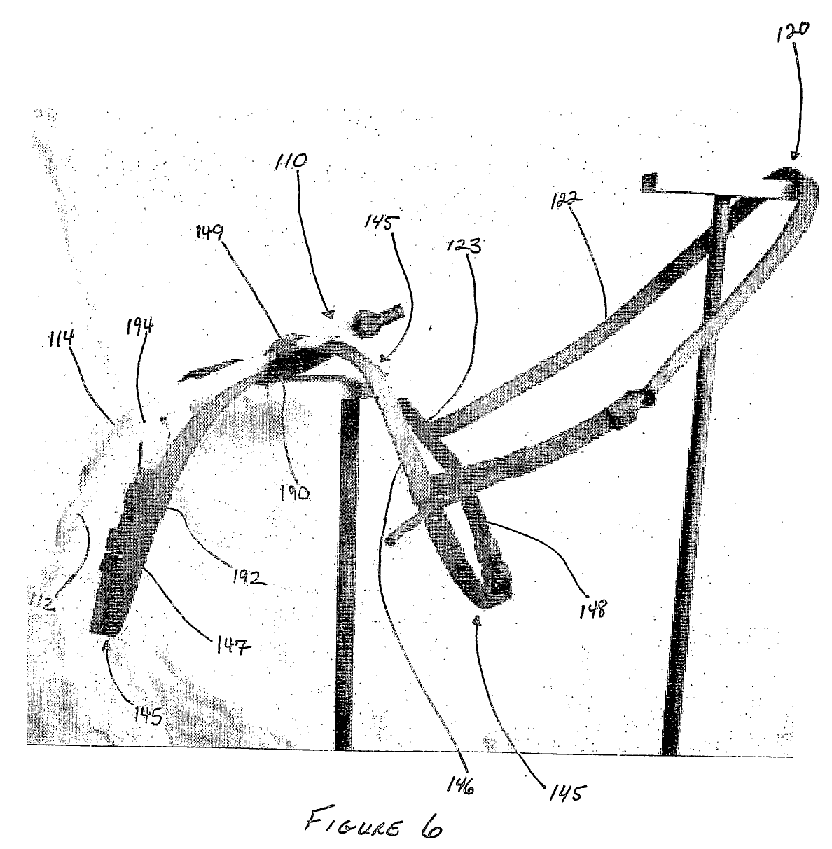

In the embodiment shown in FIGS. 6-8, the device 110 of the invention is designed such that attachment means 120 is a stand alone unit such that the device 110 and attachment means 120 can be attached to the subject equine with or without a halter, bridle or other related device. The device 110 and its adjustable attachment means 120 can be worn either under or over a halter, bridle, harness or the like.

The upper portion of attachment means 120 comprises an adjustable crown strap 122 which can be slidably attached at distal ends 123 to the lower portion of attachment means 120. The lower portion of attachment means 120 is comprised of an adjustable “figure-8” nose band assembly 145. The nose band assembly 145 is further comprised of a continuous strap 146 having two portions, an adjustable throat latch loop 148 and a chin strap portion 147. The nose band assembly 145 is designed to fit around the equine muzzle such that an “X” is formed on the bridge of the nose of the subject horse as the continuous strap 146 loops under the chin of the horse and over the nose and under the throat latch. At the point where continuous strap 146 crosses itself (on the bridge of the nose) the device 110 and especially elongated tubes 112, 114 are attached or fastened to attachment means 120 via cannula attachment straps 149 as shown best in the top plan view of FIG. 8.

In the embodiment shown in FIG. 8, a first attachment strap 149a crosses over and around elongated tubes 112,114 substantially perpendicular to the long axis thereof and a second attachment strap 149b is located between elongated tubes 112,114 crossing over elongated strap 149a and under the point at which continuous strap 146 crosses (forms an “X” in the “figure-8” configuration as set forth above) on the bridge of the nose. Optionally, a nose bridge pad 190 may be incorporated into the above-arrangement to provide additional stability and support for the attachment of the device 110 as well as to add comfort to the subject equine.

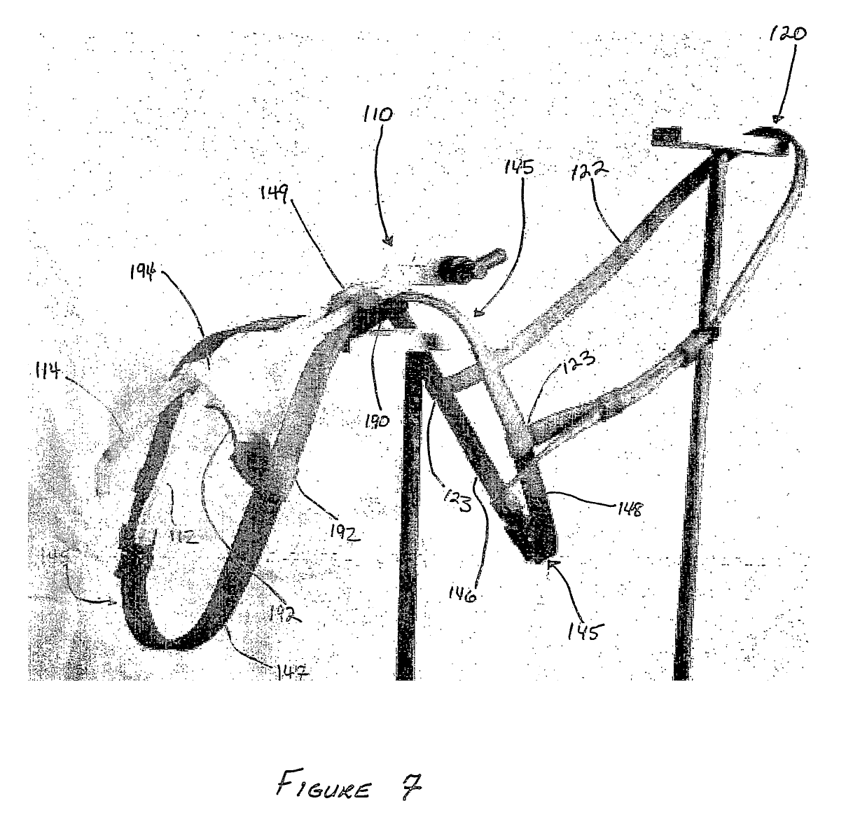

A second nose bridge band 192 may optionally be incorporated into the distal loop (chin strap portion 147) of continuous strap 146 of nose band assembly 145 for additional support of device 110 and especially to aid in securing elongated tubes 112,114 in their proper positions. As shown best in FIG. 7, nose bridge band 182 can be slidably connected to the chin strap portion 147 of continuous strap 146. In addition, velcro fasteners 184 or other type of fasteners known in the art may be added to nose bridge band 182 to hold elongated tubes 112,114 firmly into their desired position.

In another embodiment, the invention provides a method for providing inhalation therapy in an equine. As set forth above, the methods and device of the invention can be used to deliver to an equine subject any drug or other pharmaceutical agent which can be adapted for inhalation into the lungs of the subject animal. In a presently preferred embodiment, the invention provides a method for the treatment or prevention of EIPH, wherein the composition for inhalant therapy is a mixture comprised of sildenafil citrate or its congeners (e.g., metabolites, isomers, salts or other active derivatives of sildenafil citrate) using air as the delivery vehicle to carry the inhalant into the equine mammal. The sildenafil citrate or its metabolites in conjunction with the other specified elements can be nebulized into the inhalant stream at flow rates and drug delivery rates that will vary depending upon many factors, including but not limited to the relative severity of the EIPH coupled with the age, weight and gender of the equine being treated. It can be appreciated that the inhalant therapy may be administered as a pre-exercise or pre-activity treatment to lessen or eliminate the effects of exercise-induced pulmonary hemorrhage (EIPH). The inhalant therapy may also be administered post-exercise or post-activity to treat or aid in the recovery of the lung from the damage induced by EIPH in the equine athlete.

Although the presently preferred embodiment of the invention contemplates the use of the phosphodiesterase inhibitor sildenafil citrate as the preferred active for the methods set forth herein in the treatment and/or prevention of EIPH, it is specifically contemplated that other phosphodiesterase inhibitors are within the scope of the invention. Other phosphodiesterase inhibitors, including but not limited to e.g., ZAPRINAST, may be administered to the animal as an inhalant therapy, e.g., via nebulization, for the treatment of EIPH or pulmonary hypertension.

Elevations of the levels of nitric oxide created by the administration of compositions according to the methods of the invention e.g., administration of a nebulized sildenafil citrate into the pulmonary airways of horses prior to exercise, reduce the incidence of EIPH because of the effect of the released nitric oxide within the lung tissue. Since EIPH is believed to be caused by the very high blood pressures occurring in the lungs of intensely exercising horses, sildenafil citrate (or other phosphodiesterase inhibitors) induced nitric oxide dependent vasodilation acts directly to reduce the incidence and intensity of EIPH. The administration of nebulized sildenafil citrate into the airways of horses following exercise accelerates the resolution of the pathological changes in the lungs of horses because of its potent vasodilator properties.

Therefore, one method of prevention of EIPH comprises the use low levels sildenafil citrate administered as an inhalant directly into the lungs to locally elevate nitric oxide concentrations in the lungs of horses that are about to perform intense exercise and thereby avoid the potential side effects of systemic administration. This therapy regimen will allow horses to benefit from the preventive or prophylactic effects of sildenafil citrate released nitric oxide and its action against EIPH. This treatment regimen can also be provided as a therapeutic regimen following intense exercise to produce sildenafil citrate induced nitric oxide dependent vasodilation, thereby accelerating resolution of the pulmonary lesions associated with EIPH.

In one embodiment the methods of invention for the treatment and/or prevention of EIPH can comprise administration of between about 0.01 mg/kg and about 10 mg/kg of sildenafil citrate. A presently preferred embodiment, comprises administration of about 2.0 mg/kg of sildenafil citrate.

In one embodiment, the anticipated typical dosage would be based on a composition comprised of about 2.0 mg/kg combined sildenafil citrate admixed with about 500 cc sterile water and nebulized into the air flow at a rate of between about 50-200 parts per million (ppm) but especially between about 80-100 ppm for a specified period of time, e.g., for a minimum of about 2 hours per day per treatment to a maximum of about 6 hours per day depending upon the severity of the EIPH. The sterile water and sildenafil citrate represents an aqueous solution to transport the active, sildenafil citrate, and any other desired components into the horse.

Another embodiment of the methods of treatment of EIPH provided by the present invention comprises administration of compositions comprised of 1-arginine with or without sildenafil citrate. As provided by the invention, it has been found that nebulization of and administration of 1-arginine or 1-arginine precursors or analogs of 1-arginine act as an accelerant in the formation of nitric oxide in the lungs of sildenafil citrate treated horses. Additionally, it is contemplated by the invention that the methods of treatment of EIPH can further comprise nebulization of and administration of nitroglycerin and other nitric oxide donors into the lungs of horses as a means of potentiating the benefits of nitric oxide releasing compounds such as, e.g., sildenafil citrate.

Further provided by the methods of the invention are administration of a composition comprised of an iron chelating agent to reduce the pathological damage in EIPH caused the presence of free iron, released from hemoglobin in the pulmonary tissues. As part of the prophylactic and or therapeutic regimen, the methods of treatment of EIPH can comprise a composition for nebulization and administration comprised of des-ferox amine in either a prophylactic or therapeutic approach to EIPH. Des-ferox amine, by chelating iron, reduces its chemical reactivity, and thereby its ability to generate reactive oxygen species and produce the typical cumulative pathological changes associated with EIPH.

In particular, it is contemplated that the following compounds can be used either individually or in various combinations for the prophylaxis and treatment of EIPH as provided by the invention: sildenafil citrate, or a pharmaceutically acceptable derivative thereof, e.g., an isomer, a metabolite, analog or a salt, zapranist, MY5445, dipryidamole; cyclic nucleotides and their derivatives or analogs; Type V phosphodiesterase inhibitors that include but are not limited to zapranist, MY5445, dipryidamole; nitric oxide precursors including L-arginine; nitric oxide donors that include nitroglycerin, isosorbide dinitrate, erythrityl tetranitrate, amyl nitrate, sodium nitroprusside, molsidomine, linsidomine chlorhydrate, S-nitro-N-acetyl-d, 1-penicillamine, S-nitroso-N-cysteine, S-nitro-N-glutathine, diazenium diolates, and combinations thereof; nitric oxide analogs and derivatives that include nitroglycerin, nitroprusside, and Sin-1. It is also contemplated that a permeabilizing agent may be administered concurrently with the afore-mentioned therapeutic agents to facilitate the passage of the compounds through cell membranes, particularly when the compound, drug, analog, or drug derivative is a cyclic nucleotide. A preferred embodiment of the permeabilizing agent is dimethlysulfoxide (DMSO) or its derivatives or analogs. Nebulization of and administration of the afore-mentioned compounds via the devices of the invention provide multiple alternatives for effective treatment and/or prevention of EIPH in horses.

The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

What is claimed is:

1. A method for the treatment of exercise induced pulmonary hemorrhage (EIPH) in an equine, comprising administering to the equine a therapeutic amount of a composition comprised of sildenafil citrate or a pharmaceutically acceptable derivative thereof.

2. The method of claim 1, wherein the therapeutic amount comprises from between about 0.01 mg/kg and about 10 mg/kg of sildenafil citrate.

3. The method of claim 2, wherein the therapeutic amount comprises about 2.0 mg/kg of sildenafil citrate.

4. The method of claim 1, wherein the composition comprising sildenafil citrate is adapted for localized delivery into the lungs of the subject equine via nebulization, the composition being admixed with suitable liquid and administered to the equine via nebulization into an airflow stream at a rate of between about 50 to about 200 parts per million (ppm) but especially from about 80 to about 100 ppm.

5. The method of claim 4, wherein the flow rate of the air stream is between about 1 and about 4 liters per minute, but is especially between about 2 and about 3 liters per minute.

6. The method of claim 4, wherein the suitable liquid is water.

7. The method of claim 4, wherein the suitable liquid is DMSO.

8. The method of claim 1, wherein the composition further comprises DMSO.

9. The method of claim 1, wherein the composition is administered to the equine via nebulization and inhalation into the equine's lungs.

10. The method of claim 9, wherein the composition further comprises a therapeutic amount of a second phosphodiesterase inhibitor.

11. The method of claim 10, wherein the phosphodiesterase inhibitor is selected from the group consisting of zapranist, MY5445, dipryidamole and mixtures thereof.

12. The method of claim 1, wherein the composition further comprises a therapeutic amount of a nitric oxide precursor.

13. The method of claim 12, wherein the nitric oxide precursor is L-arginine.

14. The method of claim 1, wherein the composition further comprises a therapeutic amount of a nitric oxide donor.

15. The method of claim 14, wherein the nitric oxide donor is selected from the group consisting of nitroglycerin, isosorbide dinitrate, erythrityl tetranitrate, amyl nitrate, sodium nitroprusside, molsidomine, linsidomine chlorhydrate, S-nitro-N-acetyl-d, 1-penicillamine, S-nitroso-N-cysteine, S-nitro-N-glutathine, diazenium diolates, and mixtures thereof.

16. The method of claim 1, wherein the composition further comprises a therapeutic amount of a nitric oxide analog.

17. The method of claim 16, wherein the nitric oxide analog is selected from the group consisting of nitroglycerin, nitroprusside, Sin1 and mixtures thereof.

18. The method of claim 1, wherein the composition further comprises a therapeutic amount of a suitable iron chelating agent.

19. The method of claim 18, wherein the suitable iron chelating agent is des-ferox amine.

20. A method for the prevention of exercise induced pulmonary hemorrhage (EIPH) in an equine, comprising administering to the equine a prophylactic amount of a composition comprised of sildenafil citrate or a pharmaceutically acceptable derivative thereof.

21. The method of claim 20, wherein the prophylactic amount comprises from between about 0.01 mg/kg and about 10 mg/kg of sildenafil citrate.

22. The method of claim 21, wherein the propyhlactic amount comprises about 2.0 mg/kg of sildenafil citrate.

23. The method of claim 20, wherein the composition comprising sildenafil citrate is adapted for localized delivery into the lungs of the subject equine via nebulization, the composition being admixed with suitable liquid and administered to the equine via nebulization into an airflow stream at a rate of between about 50 to about 200 parts per million (ppm) but especially from about 80 to about 100 ppm.

24. The method of claim 23, wherein the flow rate of the air stream is between about 1 and about 4 liters per minute, but is especially between about 2 and about 3 liters per minute.

25. The method of claim 23, wherein the suitable liquid is water.

26. The method of claim 23, wherein the suitable liquid is DMSO.

27. The method of claim 20, wherein the composition further comprises DMSO.

28. The method of claim 20, wherein the composition is administered to the equine via nebulization and inhalation into the equine's lungs.

29. The method of claim 28, wherein the composition further comprises a prophylactic amount of a second phosphodiesterase inhibitor.

30. The method of claim 29, wherein the phosphodiesterase inhibitor is selected from the group consisting of zapranist, MY5445, dipryidamole and mixtures thereof.

31. The method of claim 20, wherein the composition further comprises a prophylactic amount of a nitric oxide precursor.

32. The method of claim 31, wherein the nitric oxide precursor is L-arginine.

33. The method of claim 20, wherein the composition further comprises a prophylactic amount of a nitric oxide donor.

34. The method of claim 33, wherein the nitric oxide donor is selected from the group consisting of nitroglycerin, isosorbide dinitrate, erythrityl tetranitrate, amyl nitrate, sodium nitroprusside, molsidomine, linsidomine chlorhydrate, S-nitro-N-acetyl-d, 1-penicillamine, S-nitroso-N-cysteine, S-nitro-N-glutathine, diazenium diolates, and mixtures thereof.

35. The method of claim 20, wherein the composition further comprises a prophylactic amount of a nitric oxide analog.

36. The method of claim 35, wherein the nitric oxide analog is selected from the group consisting of nitroglycerin, nitroprusside, Sin-1 and mixtures thereof.

37. The method of claim 20, wherein the composition further comprises a prophylactic amount of a suitable iron chelating agent.

38. The method of claim 37, wherein the suitable iron chelating agent is des-ferox amine.

39. A device for removable attachment to the head of an equine that is adapted to provide inhalation therapy of a preselected agent to the subject equine, the device comprising:
Bilateral elongated delivery tubes, each tube having a proximal end, a distal end and a body there between defining a longitudinal axis, the bilateral elongated delivery tubes being positioned relative to one another such that the body of each tube is substantially parallel along the longitudinal axis;
An inhalator manifold having a proximal end and a distal end; the distal end of the inhalator manifold being in fluid connection with the proximal ends of the elongated delivery tubes and in fluid communication with a remote drug supply means at the proximal end of the inhalator manifold; and
Attachment means for removable attachment of the device to the head of the subject equine such that the longitudinal axis of each elongated delivery tube is held in a preselected position that is substantially parallel to the bridge of the nose of the subject equine's forehead with the distal ends of the bilateral elongated delivery tubes held adjacent the external openings of the nostrils of the subject equine.

40. The device of claim 39, wherein the attachment means is a halter.

41. The device of claim 39, wherein the proximal end of the inhalator manifold is fluidly connected to the drug supply means by an elongated flexible tube.

42. The device of claim 39, wherein the drug supply means further comprises a source for providing air flow at a preselected volume and a nebulizer for introduction of the preselected agent at a predetermined concentration.

43. The device of claim 39, wherein the attachment means provides a removable attachment to a halter.

44. The device of claim 39, wherein the attachment means provides a removable attachment to a bridle.

45. The device of claim 39, wherein the body of each bilateral elongated delivery tube has an adjustment means for adjustment of the length of the body to a preselected distance.

46. The device of claim 45, wherein the adjustment means for each bilateral elongated delivery tube further comprises: a first segment of the bilateral elongated delivery tube having a distal end with an inside diameter that is sized complimentary to the outside diameter of the proximal end of a second segment such that the proximal end of the second segment is slidably and sealingly engaged with the distal end of the first segment.
